(19) United States
(12) Patent Application Publication
(10) Pub. No.: US 2016/0302880 A1
(43) Pub. Date: Oct. 20, 2016

(54) MAGETIC RESONANCE COIL ASSEMBLY FOR FIDUCIAL MARKERS

(71) Applicant: KONINKLIJKE PHILIPS N.V., EINDHOVEN (NL)

(72) Inventors: FALK UHLEMANN, EINDHOVEN (NL); SASHA KRUEGER, EINDHOVEN (NL); DANIEL WIRTZ, EINDHOVEN (NL); STEFFEN WEISS, EINDHOVEN (NL)

(21) Appl. No.: 15/100,652

(22) PCT Filed: Dec. 8, 2014
(86) PCT No.: PCT/EP2014/076807

§ 371 Date: Jun. 1, 2016

(30) Foreign Application Priority Data
Dec. 10, 2013 (EP) 13196450.4

(51) Int. Cl. A61B 90/00; A61B 5/055; G01R 33/341; G01R 33/28; G01R 33/34

(52) U.S. Cl. CPC A61B 90/39; G01R 33/285; G01R 33/34069; G01R 33/341; G01R 33/34007; A61B 5/055; A61B 2090/3958

(57) ABSTRACT

The invention provides for a medical apparatus (100) with a magnetic resonance coil assembly (102, 102′) comprising a magnetic resonance antenna with a first antenna portion (108, 108′) and a second antenna portion (110, 110′) for receiving magnetic resonance location data (1246) from a fiducial marker (118, 300, 400, 500). The magnetic resonance coil assembly further comprises a clamp with a first clamping portion (104, 104′) and a second clamping portion (106, 106′) operable for being moved between an open and a closed configuration. The first clamping portion comprises the first antenna portion. The second clamping portion comprises the second antenna portion. The first and second clamping portions are operable for securing the fiducial marker within a signal reception volume (111) in the closed configuration. When in the open position, the first and second clamping portions enable the fiducial marker being moved into or out of the signal reception volume.

### TECHNICAL FIELD

The invention relates to magnetic resonance imaging, in particular to fiducial markers in magnetic resonance imaging.

### BACKGROUND OF THE INVENTION

The availability of interactive real-time MRI and MR-conditional instruments has lead to an increasing use of MR-guidance especially in transcutaneous procedures performed with needles or linear ablation probes. Besides the lack of ionizing radiation MR-guidance offers a number of advantages for such procedures, the most important one being the soft tissue contrast and full tomographic capability of MR, if compared with CT or US. State-of-the-art clinical MR-guided percutaneous interventions use pre-operative 3D MR images to plan the device path, then stereotactic devices are used as guides to align the device with the target and to guide its insertion, which is mostly performed outside the MR bore. Finally, MR is used to confirm that the device has reached the target.

Because stereotactic procedures are prone to registration errors due to patient motion and needle bending, and because they involve a complicated workflow (patient movement into and out of bore), advanced centers are now practicing so-called free-hand procedures, in which the device is advanced without any physical stereotactic device guide under real-time image guidance inside the MR. This is facilitated by dedicated MR sequences that visualize the target lesion and the device with high conspicuity and by the availability of open MR systems

In Coutts et. at. “Integrated and Interactive Position Tracking and Imaging of Interventional Tools and Internal Devices Using Small Fiducial Receiver Coils,” Magnetic Resonance in Medicine, vol. 40, 1998, pages 908-913, a method of tracking the position of a rigid device within a magnetic resonance scanner is disclosed. The position tracking is performed by means of two or three small magnetic resonance receiver coils attached to individual receiver channels.

The U.S. Pat. No. 5,307,806 concerns an NMR pelvic coil with two pivotally connected posterior and anterior segments. In an open position the patient's pelvis is moved into the space between the segments. IN the closed position, the segments fit closely around the patient's pelvis.

International patent application publication WO2012/137148 A1 discloses a magnetic resonance fiducial marker which comprises a magnetic resonance receive coil surrounding a toroidal magnetic resonance signal volume.

International patent application publication WO 2007/046011 A1 discloses a system for tracking a fiducial marker assembly in a magnetic resonance imaging scanner.

### SUMMARY OF THE INVENTION

The invention provides for a medical apparatus in the independent claim. Embodiments are given in the dependent claims.

As will be appreciated by one skilled in the art, aspects of the present invention may be embodied as an apparatus, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer executable code embodied thereon.

Any combination of one or more computer readable medium(s) may be utilized. The computer readable medium may be a computer readable signal medium or a computer readable storage medium. A ‘computer-readable storage medium’ as used herein encompasses any tangible storage medium which may store instructions which are executable by a processor of a computing device. The computer-readable storage medium may be referred to as a computer-readable non-transitory storage medium. The computer-readable storage medium may also be referred to as a tangible computer readable medium. In some embodiments, a computer-readable storage medium may also be able to store data which is able to be accessed by the processor of the computing device. Examples of computer-readable storage media include, but are not limited to: a floppy disk, a magnetic hard disk drive, a solid state hard disk, flash memory, a USB thumb drive, Random Access Memory (RAM), Read Only Memory (ROM), an optical disk, a magneto-optical disk, and the register file of the processor. Examples of optical disks include Compact Disks (CD) and Digital Versatile Disks (DVD), for example CD-ROM, CD-RW, CD-R, DVD-ROM, DVD-RW, or DVD-R disks. The term computer readable-storage medium also refers to various types of recording media capable of being accessed by the computer device via a network or communication link. For example a data may be retrieved over a modem, over the internet, or over a local area network. Computer executable code embodied on a computer readable medium may be transmitted using any appropriate medium, including but not limited to wireless, wire line, optical fiber cable, RF, etc., or any suitable combination of the foregoing.

A computer readable signal medium may include a propagated data signal with computer executable code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

‘Computer memory’ or ‘memory’ is an example of a computer-readable storage medium. Computer memory is any memory which is directly accessible to a processor. ‘Computer storage’ or ‘storage’ is a further example of a computer-readable storage medium. Computer storage is any non-volatile computer-readable storage medium. In some embodiments computer storage may also be computer memory or vice versa.

A ‘processor’ as used herein encompasses an electronic component which is able to execute a program or machine executable instruction or computer executable code. References to the computing device comprising “a processor” should be interpreted as possibly containing more than one processor or processing core. The processor may for instance be a multi-core processor. A processor may also refer to a collection of processors within a single computer system or distributed amongst multiple computer systems. The term computing device should also be interpreted to possibly refer to a collection or network of computing devices each comprising a processor or processors. The computer executable code may be executed by multiple processors that may be within the same computing device or which may even be distributed across multiple computing devices.

Computer executable code may comprise machine executable instructions or a program which causes a processor to perform an aspect of the present invention. Computer executable code for carrying out operations for aspects of the present invention may be written in any combination of one or more programming languages, including an object oriented programming language such as Java, Smalltalk, C++ or the like and conventional procedural programming languages, such as the “C” programming language or similar programming languages and compiled into machine executable instructions. In some instances the computer executable code may be in the form of a high level language or in a pre-compiled form and be used in conjunction with an interpreter which generates the machine executable instructions on the fly.

The computer executable code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described with reference to flowchart illustrations and/or block diagrams of methods, apparatus (systems) and computer program products according to embodiments of the invention. It will be understood that each block or a portion of the blocks of the flowchart, illustrations, and/or block diagrams, can be implemented by computer program instructions in form of computer executable code when applicable. It is further understood that, when not mutually exclusive, combinations of blocks in different flowcharts, illustrations, and/or block diagrams may be combined. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

A ‘user interface’ as used herein is an interface which allows a user or operator to interact with a computer or computer system. A ‘user interface’ may also be referred to as a ‘human interface device.’ A user interface may provide information or data to the operator and/or receive information or data from the operator. A user interface may enable input from an operator to be received by the computer and may provide output to the user from the computer. In other words, the user interface may allow an operator to control or manipulate a computer and the interface may allow the computer indicate the effects of the operator's control or manipulation. The display of data or information on a display or a graphical user interface is an example of providing information to an operator. The receiving of data through a keyboard, mouse, trackball, touchpad, pointing stick, graphics tablet, joystick, gamepad, webcam, headset, gear sticks, steering wheel, pedals, wired glove, dance pad, remote control, and accelerometer are all examples of user interface components which enable the receiving of information or data from an operator.

A ‘hardware interface’ as used herein encompasses an interface which enables the processor of a computer system to interact with and/or control an external computing device and/or apparatus. A hardware interface may allow a processor to send control signals or instructions to an external computing device and/or apparatus. A hardware interface may also enable a processor to exchange data with an external computing device and/or apparatus. Examples of a hardware interface include, but are not limited to: a universal serial bus, IEEE 1394 port, parallel port, IEEE 1284 port, serial port, RS-232 port, IEEE-488 port, Bluetooth connection, Wireless local area network connection, TCP/IP connection, Ethernet connection, control voltage interface, MIDI interface, analog input interface, and digital input interface.

A ‘display’ or ‘display device’ as used herein encompasses an output device or a user interface adapted for displaying images or data. A display may output visual, audio, and or tactile data. Examples of a display include, but are not limited to: a computer monitor, a television screen, a touch screen, tactile electronic display, Braille screen, Cathode ray tube (CRT), Storage tube, Bistable display, Electronic paper, Vector display, Flat panel display, Vacuum fluorescent display (VF), Light-emitting diode (LED) displays, Electroluminescent display (ELD), Plasma display panels (PDP), Liquid crystal display (LCD), Organic light-emitting diode displays (OLED), a projector, and Head-mounted display.

Magnetic Resonance (MR) data is defined herein as being the recorded measurements of radio frequency signals emitted by atomic spins by the antenna of a Magnetic resonance apparatus during a magnetic resonance imaging scan. Magnetic resonance data is an example of medical image data. A Magnetic Resonance Imaging (MRI) image is defined herein as being the reconstructed two or three dimensional visualization of anatomic data contained within the magnetic resonance imaging data. This visualization can be performed using a computer.

Magnetic resonance location data as used herein encompasses magnetic resonance data that is acquired for determining the location of a fiducial marker.

In one aspect the invention provides for a medical apparatus comprising a magnetic resonance coil assembly. The magnetic resonance coil assembly comprises a magnetic resonance antenna comprising a first antenna portion and a second antenna portion for receiving magnetic resonance location data from the fiducial marker. In some examples the first and second antenna portions may be antenna elements. In other examples the first and second antenna portions may be parts of an antenna that are assembled into or connected to form a single antenna element. A fiducial marker as used herein encompasses an object which may be placed into the field of view of a magnetic resonance imaging system and which appears in a magnetic resonance image which is produced or reconstructed from magnetic resonance data. The fiducial marker is for use as a point of reference or as a point of measure.

The magnetic resonance coil assembly comprises a clamp. The clamp comprises a first clamping portion and a second clamping portion. The first clamping portion and the second clamping portion are operable for being moved between an open configuration and a closed configuration. The first clamping portion comprises the first antenna portion. The second clamping portion comprises the second antenna portion. When in the closed configuration the first clamping portion and the second clamping portion are operable for securing the fiducial marker within a signal reception volume between the first antenna portion and the second antenna portion. When in the open position the first clamping portion and the second clamping portion are operable to enable the fiducial marker being moved into or out of the signal reception volume. This embodiment may be beneficial because it provides for a magnetic resonance coil assembly which can be clamped onto a fiducial marker. This keeps the magnetic resonance antenna separate from the fiducial marker. A coil as used herein may be interpreted as an antenna. In the magnetic resonance imaging technology the term coil is typically used in place of the term antenna.

The fiducial marker may contain a signal emitting substance when magnetic resonance imaging is performed. For instance a fiducial marker may have a tube or other container filled with a liquid or material which shows up in a magnetic resonance image. The magnetic resonance antenna functions as a local antenna which is placed about the fiducial marker. The fiducial marker may also be referred to as a magnetic resonance fiducial marker. The fiducial marker may comprise a signal volume. The signal volume may contain a magnetic resonance signal emitting substance. The signal volume may in some examples be toroidal. The signal volume may in other examples be partially toroidal with a break or open region in part of the toroid.

In another embodiment the magnetic resonance coil assembly further comprises a transmitter operable for receiving a magnetic resonance signal from the magnetic resonance antenna and transmitted to the magnetic resonance imaging system. In various examples the term transmitter may be interpreted differently. In some cases this may refer to an optical transmission device and fiber optics may be used for transmitting the data to the magnetic resonance imaging system.

In other examples the transmitter may function wirelessly. For instance a Wi-Fi, a Bluetooth or other radio transmission standard could be used. Particularly in the case of the wireless transmitter this may be beneficial because it may reduce the number of wires necessary to use the magnetic resonance antenna. For instance if a physician is using the medical apparatus to guide a catheter using the fiducial markers and one or more magnetic resonance antennas then using a number of wires may facilitate the use of the catheter.

In another embodiment the first antenna portion is a first saddle coil and the second antenna portion is a second saddle coil.

In this embodiment the two saddle coils may straddle the fiducial marker and allow for a good magnetic resonance signal reception from the fiducial marker.

In another embodiment the first clamping portion comprises a first electrical contact connected to the first antenna portion. The second clamping portion comprises a second electrical contact connect to the second antenna portion. The clamp is operable for connecting the first electrical contact to the second electrical contact to form an electrical connection. The first antenna portion and the second antenna portion are operable to form a single surface coil. This embodiment may be beneficial because it enables the surface coil to be conveniently placed around the fiducial marker.

In another embodiment the magnetic resonance coil assembly further comprises a fiducial marker sensor system for sensing the fiducial marker.

In another embodiment the fiducial marker sensor system comprises any one of the following: a switch for sensing if the clamp is closed in the closed configuration, an impedance measurement system for measuring an impedance of the magnetic resonance antenna to determine if the fiducial marker is within the signal reception volume and/or determine a type of the fiducial marker, and combinations thereof. This embodiment may be beneficial because it may help to ensure that the fiducial marker is inserted properly into the magnetic resonance coil assembly.

In another embodiment the medical apparatus further comprises an indicator operable for displaying the signal if the fiducial marker sensor system senses the fiducial marker. This may be beneficial because an operator or physician using the magnetic resonance coil assembly can conveniently know if the fiducial marker is properly inserted into the magnetic resonance coil assembly.

In another embodiment the medical apparatus further comprises the fiducial marker.

In another embodiment the fiducial marker comprises a shaft of a medical device or is operable for receiving the shaft. This embodiment may be beneficial because the location of a shaft or inserter or catheter can be determined using the medical apparatus.

In another embodiment the clamp is operable for securing the shaft to the magnetic resonance coil assembly when in the closed configuration. For instance when the magnetic resonance coil assembly is closed, it may clamp down or grip the shaft.

In another embodiment the fiducial marker comprises a hole for the shaft. The fiducial marker is toroidal. The fiducial marker comprises a tube filled with the magnetic resonance detectable substance surrounding the shaft. The tube had a gap. The shaft is operable for being removed at a right angle to the hole through the gap. This embodiment may be beneficial because after for instance the insertion of a catheter it may be desired to remove the fiducial marker.

In another embodiment the fiducial marker comprises an adhesive for attaching to an object. For instance the object may be the subject. Placing the fiducial marker on an object or the subject may be beneficial because it may be useful for determining the entry point into the object or the subject.

In another embodiment the medical instrument comprises an interventional device.

In another embodiment the interventional device comprises the fiducial marker.

The fiducial marker may be attached or permanently attached to the interventional device.

The fiducial marker may contain a toroidally shaped signal volume in some embodiments. This may enable measurement of the position and/or orientation of the needle axis with only one or two markers but without blocking the needle axis as would be the case for point-like markers. Hence, embodiments of the invention may be compatible with any needle-type device and, additionally, secondary devices can be introduced, e.g. a stylet or biopsy device into a hollow needle.

In another embodiment the interventional device is a needle.

In another embodiment the interventional device is a linear ablation probe.

In another embodiment the interventional device is a cryoprobe. A cryoprobe supplies cryogenic fluid or cools a vicinity of the probe tip to cryogenic temperatures to cool tissues to the point of ablation.

In another embodiment the interventional device is a laser ablation probe.

In another embodiment the interventional device is a biopsy needle.

In another embodiment the interventional device is a hollow needle.

In another embodiment the interventional device is a microwave probe. The microwave probe is adapted for delivering microwave energy to tissue in the vicinity of the tip of the shaft.

In another embodiment the interventional device is a guide wire delivery system. The guide wire may for instance be delivered using a hollow needle or other structure. The guide wire may then be used to deliver another interventional apparatus to the target zone.

In another embodiment the medical apparatus further comprises a magnetic resonance imaging system for acquiring magnetic resonance data from a subject. The medical apparatus further comprises a medical device comprising a shaft. The shaft is adapted for being inserted into the subject. The fiducial marker is operable for being attached to the shaft. The medical apparatus further comprises a processor for controlling the medical apparatus. The medical apparatus further comprises a memory for storing machine-executable instructions for execution by the processor. Execution of the instructions causes the processor to acquire the magnetic resonance data. Execution of the instructions further causes the processor to reconstruct the magnetic resonance data into a magnetic resonance image. Execution of the instructions furthers cause the processor to receive the selection of a target volume within the magnetic resonance image.

Execution of the instructions further causes the processor to repeatedly acquire the magnetic resonance location data from the magnetic resonance antenna. The magnetic resonance location data is descriptive of the location of the first magnetic resonance fiducial marker. Execution of the instructions further cause the processor to render a view of the magnetic resonance data indicating the position of the shaft relative to the target zone on a display device. The view is determined using at least the location data and the location of the target volume.

This embodiment may be beneficial because it enables the medical apparatus to adjust the view of the image data for the magnetic resonance imaging system such that the shaft is conveniently displayed.

In other embodiments or examples the medical apparatus may comprise multiple magnetic resonance antennas each which supply data to the magnetic resonance imaging system. The magnetic resonance apparatus and the clamp may also have or comprise multiple fiducial markers for putting into the multiple magnetic resonance antennas.

It is understood that one or more of the aforementioned embodiments of the invention may be combined as long as the combined embodiments are not mutually exclusive.

### BRIEF DESCRIPTION OF THE DRAWINGS

In the following preferred embodiments of the invention will be described, by way of example only, and with reference to the drawings in which:

FIG. 1 illustrates an example of a magnetic resonance coil assembly;

FIG. 2 illustrates an example of a fiducial marker;

FIG. 3 illustrates a further example of a fiducial marker;

FIG. 4 illustrates a further example of a fiducial marker;

FIG. 5 illustrates a further example of a fiducial marker;

FIG. 6 illustrates a further example of a magnetic resonance coil assembly;

FIG. 7 illustrates a further example of a magnetic resonance coil assembly;

FIG. 8 illustrates a further example of a magnetic resonance coil assembly;

FIG. 9 illustrates an example of a magnetic resonance antenna circuit;

FIG. 10 illustrates a further example of a magnetic resonance coil assembly;

FIG. 11 illustrates a further example of a magnetic resonance coil assembly;

FIG. 12 illustrates an example of a medical apparatus;

FIG. 13 shows a flow chart illustrating a method of operating the medical apparatus of FIG. 12; and

FIG. 14 shows a flow chart illustrating an alternative method of operating the medical apparatus of FIG. 12.

### DETAILED DESCRIPTION OF THE EMBODIMENTS

Like numbered elements in these figures are either equivalent elements or perform the same function. Elements which have been discussed previously will not necessarily be discussed in later figures if the function is equivalent.

An example of a tracking device, which may comprise a fiducial marker, and method for MR-guided interventions is described. It may comprise of a small clamp-on device (also referred to herein as a clamp) equipped with two saddle-shaped active marker coils used in combination with a passive marker or fiducial marker which is possibly of toroidal shape. The fiducial marker provides a Magnetic Resonance (MR) signal volume. In some examples two of these tracking devices are placed on the axis of the interventional instrument (e.g. biopsy needle).

The toroidal shape of the passive markers allows measurement of the position and orientation of any needle-type instrument without compromising its functionality and without obstructing its axis or back-loading capability. Hence, secondary devices can be introduced, e.g. a stylett or biopsy device into a hollow needle.

The clamp-on mechanism of the tracking device may allow:
easy placement and removal at any point during the intervention
alignment to and fixation to the axis of the interventional device
fixation of the passive marker.

The small size and low weight of the tracking device permits uncompromised maneuverability and minute haptic feedback as required when advancing the needle device into the patient.

Low cost price disposable passive markers and accessory parts for sterility are disclosed. A similar prototype system implementation is WiP.

Availability of interactive real-time MRI and MR-conditional instruments has lead to an increasing use of MR-guidance especially in transcutaneous procedures performed with needles or linear ablation probes. Besides the lack of ionizing radiation, MR-guidance offers a number of advantages for such procedures, the most important one being the soft tissue contrast and full tomographic capability of MR, if compared with CT or US. State-of-the-art clinical MR-guided percutaneous interventions use pre-operative 3D MR images to plan the device path, then stereotaxy devices are used to align the device with the target and to guide its insertion, which is mostly performed outside the MR bore. Finally, MR is used to confirm that the device has reached the target.

Because stereotactic procedures are prone to registration errors due to tissue movement/deformation and needle bending, and because they involve a complicated workflow (patient into and out of bore), advanced centers are now practicing procedures where the device is advanced under live real-time image guidance inside the MR. This is facilitated by dedicated MR sequences that visualize the target lesion and the device with high conspicuity and by the availability of wide-bore and open magnet MR systems.

However, this approach requires alignment of the imaging slices with needle and/or target lesion. Manual adjustment of slices is current practice, but requires that the interventionist communicates the requested slice adjustments to the MR operator outside the MR room, which is not trivial and requires an experienced and well attuned team. Means to support, automate, and improve the workflow of such free-hand interventions are mandatory to foster a wide-spread use.

For automatic adaptation of the scan planes, the technology of active markers for position tracking of devices and respective automatic scan plane definition may be used. Recently, a hand-held, actively tracked needle guidance tool with respective MR system software modifications to allow for simple-to-use, fast and accurate scan plane controlling was demonstrated. The needle guidance tool is directly connected to the MR system and can be used to control real-time MR imaging for optimal guidance and navigation.

Existing fiducial marker and antenna combinations may have the following disadvantage: During the interventional procedure tracking of the interventional devices is not required all the time. Though the tracking devices are much smaller than previous designs they can still hamper handling of the devices or pose a trip-risk as they cannot be removed. Introducing plugs to disconnect them during idle times is a difficult compromise between reliable mechanical/electrical connection and easy detachability and increases the cost of the (disposable) device.

A small light-weight clamp-on device or magnetic resonance coil assembly as described herein may be equipped with two saddle-shaped coils (or other types of coils) which can be clamped on a toroidal-shaped passive marker. The marker and the clamp-on device are placed on the interventional device (e.g. needle) and thereby allow to localize or locate the respective point on the needle axis with a single marker channel and irrespective of the orientation of the guide with respect to BO. Two of such tracking devices with a known spatial relation may allow to defer position and orientation of the e.g. needle axis. The clamp-on mechanism may allow easy removal and reattachment of the tracking device as needed.

With the known position and angulation of the interventional device, visualization of the device and corresponding image planes allows simplification of the above described workflow.

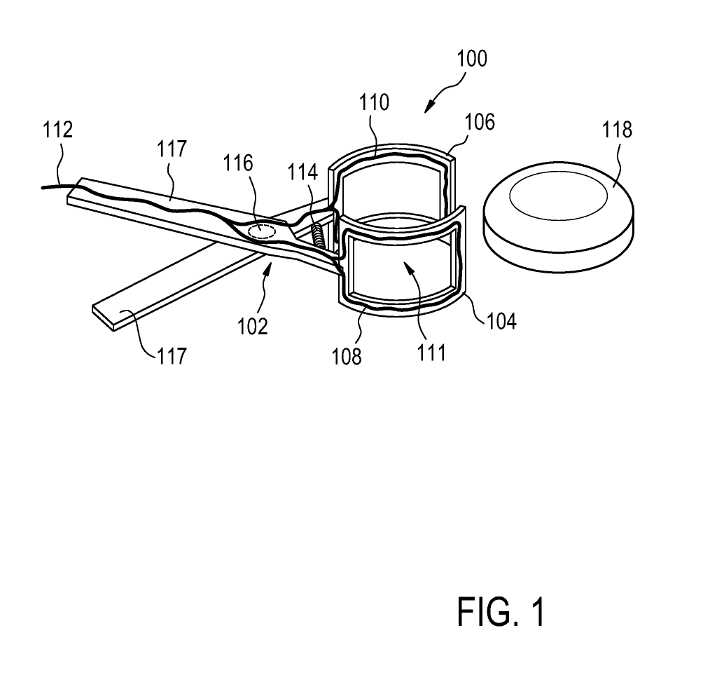

An example of a tracking device or magnetic resonance coil assembly may consist of a light-weight clamp-on device containing the active saddle-shaped coils in the two clamp halves and a passive marker providing a signal volume (e.g. a commercially available adhesive skin marker) as sketched in FIG. 1.

FIG. 1 shows an example of a medical apparatus 100. The medical apparatus is shown as comprising a magnetic resonance coil assembly 102. The magnetic resonance coil assembly has a first clamping portion 104 and a second clamping portion 106. The first clamping portion 104 and the second clamping portion 106 are tongue-shaped. The first clamping portion 104 has a first antenna portion 108 and the second clamping portion 106 has a second antenna portion 110. The first antenna portion 108 and the second antenna portion 110 can both be seen to be saddle coils. The region between the saddle coils 108, 110 forms a signal reception volume. The saddle coils 108, 110 are connected to an antenna connection 112. The antenna connection 112 for instance can be connected to the radio-frequency receiver of a magnetic resonance imaging system. There is shown an elastic portion 114 which pulls the first clamping portion 104 towards the second clamping portion 106. The pivot 116 allows the two clamping portions 104, 106 to rotate about a pivot and be pulled together by the elastic portion 114.

Each of the clamping portions 104, 106 is connected to a handle 117. By squeezing the handles 117 the magnetic resonance coil assembly 102 is brought into the open position and a fiducial marker 118 can be inserted between the two tongue-shaped clamping portions 104, 106. In this example there is an open space which the fiducial marker 118 can fit into. However, in other designs there may be slots or grooves which are fitted to accommodate the fiducial marker 118.

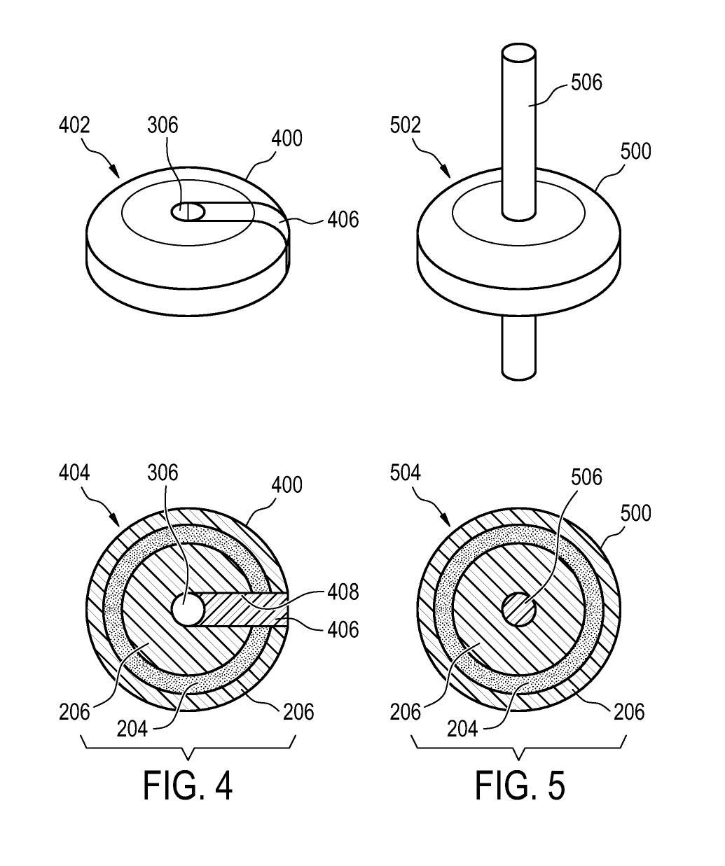

The passive marker may have a central opening to let the instrument (e.g. needle) pass. In this case the passive marker is positioned on the needle, and the clamp-on device (which is connected to the MR system) clamped on it only when active tracking is needed (see FIG. 4). The clamp-on device fixes and aligns itself and the passive marker through the clamping force. The passive marker has a known geometry and can be closely surrounded by the inner shell of the clamp.

Alignment/fixation notches along the clamp's central axis (e.g. diamond shaped to be adapted to different diameters and increase friction to clamped device) centre and fix the tracking device on the needle.

If at least one side wall of the clamp's shell is sufficiently thin and the alignment notch big enough, the clamp can also be placed on a passive marker being attached to the patient's skin to define the entry point while permitting easy insertion of the needle.

In addition to the basic tracking functionality, sensing the clamp opening (e.g. by a mechanical switch or by a change of the amount of MR or impedance signal detected) could be used to control aspects of the interventional setup e.g. start/stop image acquisition; detect different clamped device types and modify their calibration or visualization. An immediate sensing capability resides in the option to perform MR measurements for detecting tracking SNR. An open/detached clamp would have no signal and is therefore detectable.

Different mechanical inserts for the central alignment and fixation notch section could be adapted such that they specifically fit to certain devices (e.g. needles of different diameters) or attachment points including markers. These inserts can be attached to the clamp device (and open up as the clamp is opened) or to the interventional device to be tracked. This “mechanical device identification” can be used to keep spatial reference to numerous other devices which are described by their respective geometrical model and consequently transformed coordinate system.

In another example a non-rotation-symmetric attachment point (and clamp insert) in conjunction with a non-rotation-symmetric marker volume would allow to track devices with just one marker and/or its rotation around the longitudinal device axis.

The above examples were based on a passive marker with a toroidal shape. Alternatively, this toroid may have a gap at one side so that it can be clamped onto and removed from the needle while the needle can stay in place.

Ultimately, use of a passive marker as separate part may be omitted completely. Instead, signal volumes may be integrated into the saddle coils of the clamp-on device. This allows quick removal of the entire tracking device at the cost of losing the option to find the needle position passively at all times.

The passive markers may be manufactured and wrapped as sterile single-use devices. Use of the clamp-on device in a sterile environment is enabled by providing sterile dedicated single-use plastic drapes that can be wrapped over the clamp-on device before it is clamped on the needle. The shape of the drapes is adapted to closely fit the shape of the clamp-on device.

The real-time tracking device is small, light-weight, and may be equipped with minimal wiring.

It can be aligned and fixated on as well as easily removed from devices to be tracked as required by clinical workflow/intervention stage.

The passive markers and the sterile drapes are implemented as single-use devices, enabling to generate device-based revenues.

The invention may can be applied to all MR-guided interventions performed with linear-shaped (or in other described embodiments arbitrarily shaped) devices.

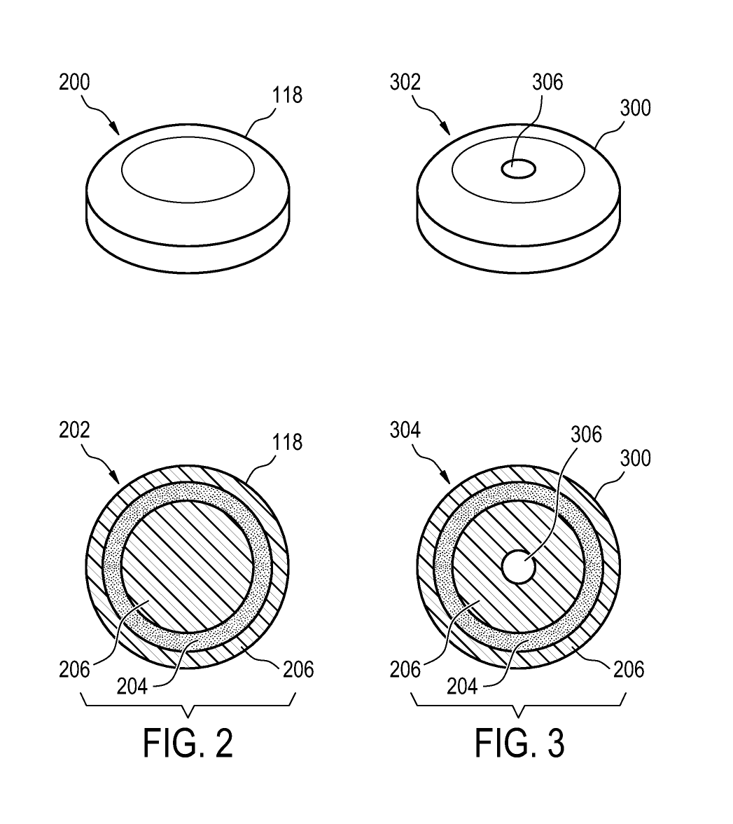

FIG. 2 shows a perspective view 200 and a cross-sectional view 202 of the fiducial marker 118. In the cross-sectional view 202 it can be seen if there is a tube of MR signal emitting substance 204 that is encased in an encasing material 206. For instance the encasing material 206 may be a plastic. The MR emitting substance 204 may be for instance water or fat or other material which may be picked up by the particular magnetic resonance protocol being used.

FIG. 3 shows a further example of a fiducial marker 300. The fiducial marker 300 is shown in a perspective view 302 and a cross-sectional view 304. The fiducial marker 300 is similar to that shown in FIG. 2 except there is a through hole 306 which is operable for receiving a spherical or shaft-shaped object. The fiducial marker 300 may be placed onto the shaft of a medical instrument or device. This may be useful for locating the position of a medical instrument or tool when used in a procedure during magnetic resonance imaging.

FIG. 4 shows a further example of a fiducial marker 400. The fiducial marker 400 is similar to that shown in FIG. 3, however there is additionally a removable plug 406. There is a gap 408 in the MR signal emitting substance 204. Instead of being solid there is a removable plug 408 that can be slid out to allow a shaft to be removed from the hole 306 in a direction perpendicular to the axis of the shaft. This embodiment may be beneficial if it is desired to remove a medical instrument after it has been positioned or used. For instance after a catheter has been inserted it may be inconvenient to remove the catheter again to take off the fiducial marker 400. This enables a fiducial marker 400 to be easily removed without moving the shaft.

FIG. 5 shows a further example of a fiducial marker 500. The fiducial marker 500 is similar to that shown in FIG. 3. However, within the hole there is permanently mounted a shaft 506. For instance medical instruments may come with a fiducial marker 500 pre-attached and positioned.

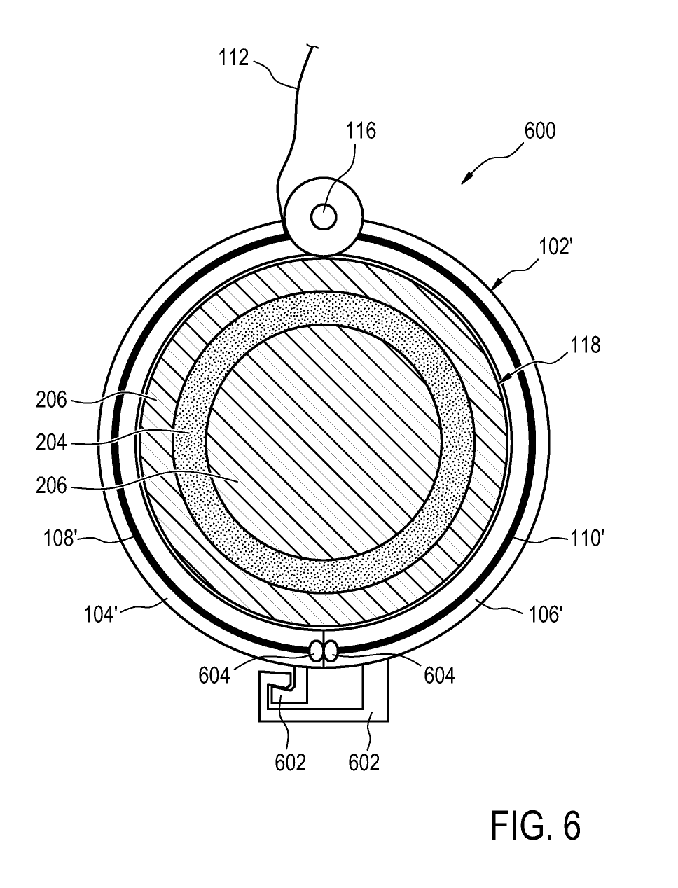

FIG. 6 shows a further example of a medical apparatus. The medical apparatus 600 is shown as comprising a magnetic resonance coil assembly 102′. The magnetic resonance coil assembly 102 comprises a first clamping portion 104′ and a second clamping portion 106′. There is again a first antenna portion 108′ embedded in the first clamping portion 104 and a second antenna portion 110 embedded in the second clamping portion 106′. The antenna in this example is different from that shown in FIG. 1. In this case the antenna portions 108′, 106′ form a surface coil around the fiducial marker 118. There is a latch 602 that holds the two clamping portions 104′, 106′ together. There is a first electrical contact 604 on an end of the first antenna portion 108′ and a second electrical contact 606 on another end of the second antenna portion 110′. The clamp 602 presses the first and second electrical contacts 604, 606 together. When in a closed position the first antenna portion 108′ and the second antenna portion 110′ form a single surface coil or antenna about the fiducial marker 118. The first coil can be connected to a radio-frequency receiver of a magnetic resonance imaging system using the lead 112. The two clamping portions 104′, 106′ are shown as being hinged by a pivot 116.

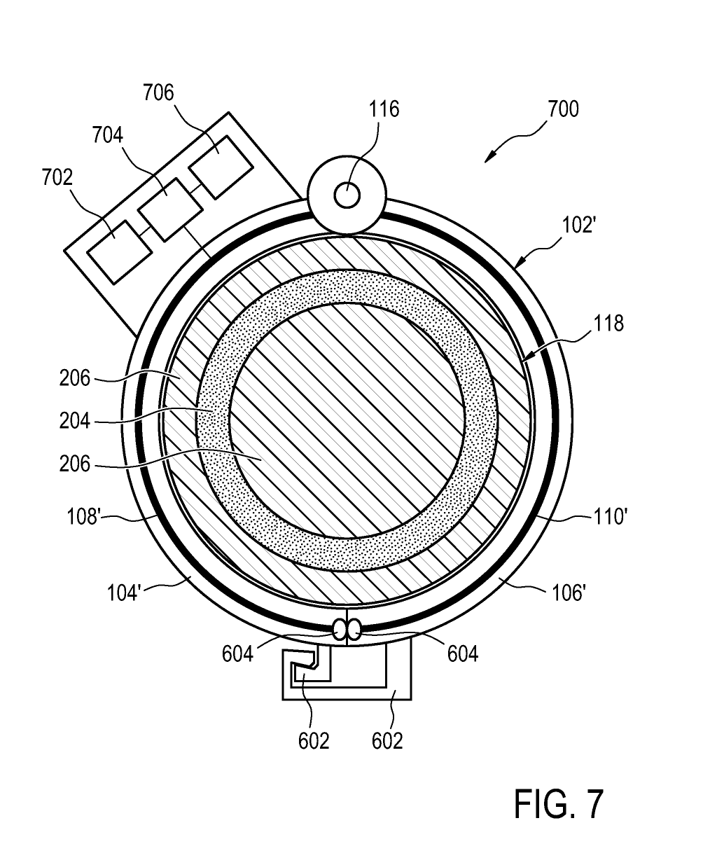

FIG. 7 shows a further example of a medical apparatus 700. The medical apparatus 700 is similar to the medical apparatus 600 shown in FIG. 6. However, instead of a lead 112 connecting to a receiver the fiducial marker 700 has a receiver 704 which is connected directly to the surface coil 108′, 110′. There is a battery 702 for powering the receiver 704 and a transmitter 706. The transmitter 706 takes the signal received by the receiver 704 and re-transmits it to a magnetic resonance imaging system. The battery 702 may be replaced by a cable supplying power or any other means of energy harvesting. The receiver 704 essentially digitizes the signal on the surface coil 108′, 110′ and then the transmitter 706 uses a protocol to transmit it to the magnetic resonance imaging system. The transmitter 706 may be for instance a Wi-Fi or Bluetooth transmitter or other radio-frequency transmission system, it may also be transmitted optically for instance via a fiber optics. The receiver and transmitter arrangement shown in FIG. 7 may also be applied to other embodiments such as that shown in FIG. 1.

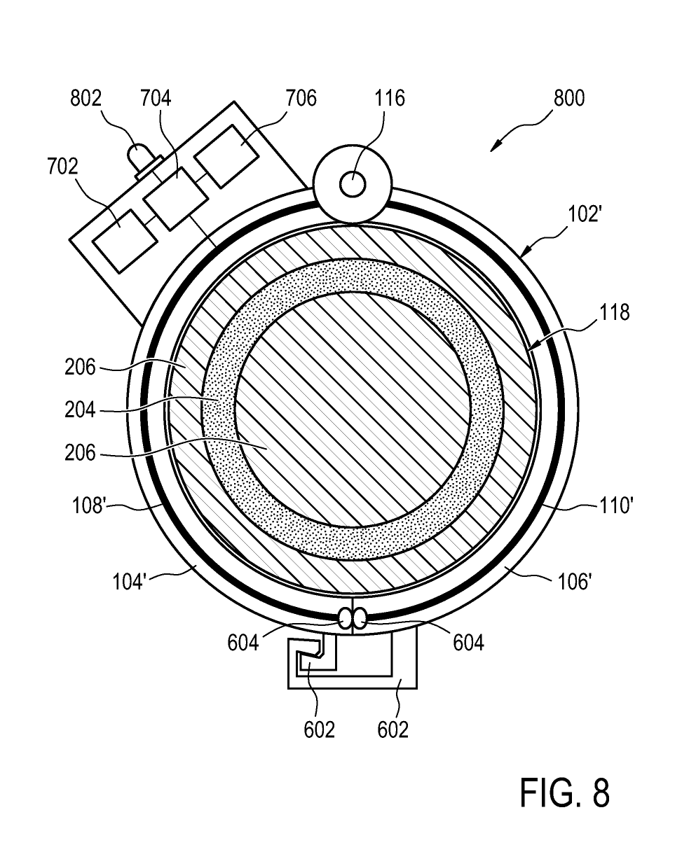

FIG. 8 shows a further example of a medical apparatus 800. The medical apparatus 800 shown in FIG. 8 is very similar to that shown in FIG. 7. However, there is additionally a visual indicator 802. For instance there may be a switch embedded which is closed when a fiducial marker 118 is properly installed. Alternately the impedance of the surface coil 108′, 110′ may also be altered if fiducial marker 118 is present. For instance the receiver 704 could be replaced by a transceiver which is able to measure the impedance of the surface coil 108′, 110′. When a fiducial marker 118 is detected then the visual indicator 802 may be lit to indicate to an operator that the fiducial marker 118 is properly installed. Such an indicator 802 may also be used with the example shown in FIG. 1.

In FIG. 1, FIG. 6, FIG. 7 and FIG. 8 any of the fiducial markers illustrated or described in this application may be used. Additionally the fiducial marker shown in FIGS. 2-5 may also have an adhesive layer on one side to attach to an object or to the surface of a subject.

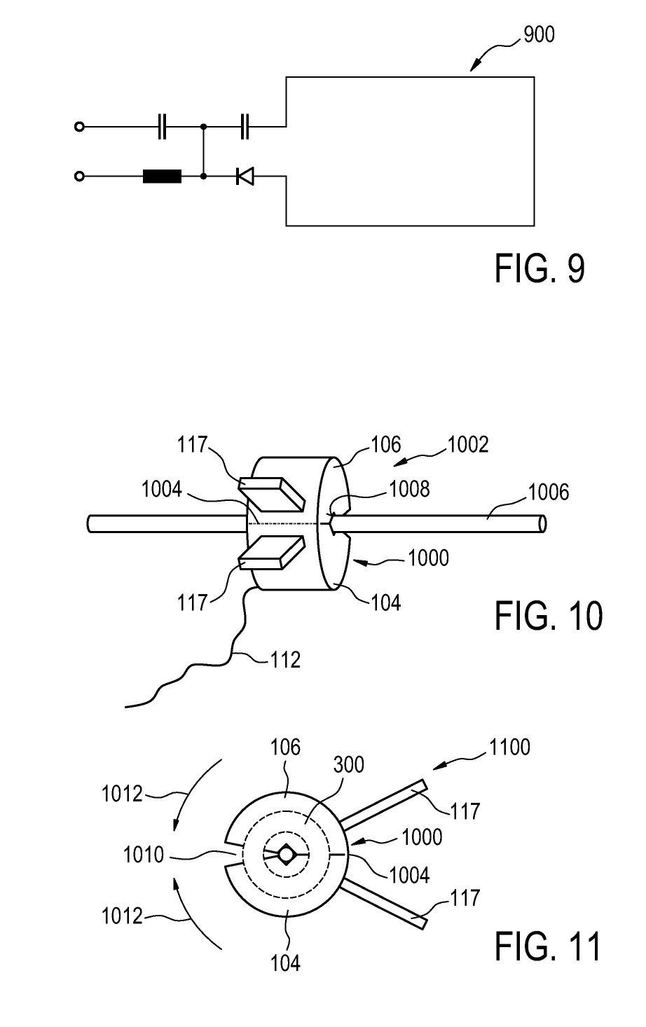

FIG. 9 shows an example of a schematic 900 of a magnetic resonance antenna circuit. FIGS. 10 and 11 show a further example of a medical apparatus 1000.

FIG. 10 shows a perspective view 1002 and FIG. 11 shows a top view 1100 of a further example of a magnetic resonance coil assembly 1000. The mechanism is similar to that shown in FIG. 1. However, there is not an open space around the saddle coils. The two clamping portions 104, 106 are joined by a hinge 1004. Clamping portions 104, 106 clamp down on a shaft 1006 and a fiducial marker 300. There is a diamond-shaped alignment and fixation notch 1008. There is a gap 1010 between the two clamping portions 104, 106. The fiducial marker 300 is partially surrounded by internal saddle coils. The arrows 1012 mark the direction of closing forces exerted by an internal spring.

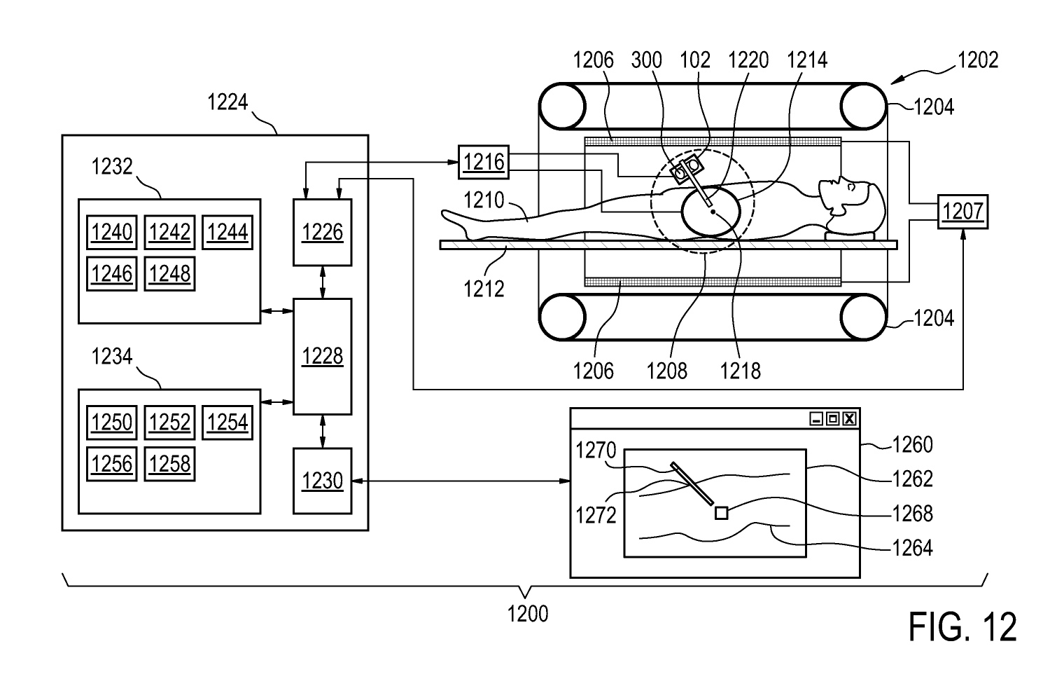

FIG. 12 shows a medical apparatus 1200 according to an embodiment of the invention. The medical apparatus 1200 comprises a magnetic resonance imaging system 1202. The magnetic resonance imaging system 1202 comprises an open magnet 1204. In the open magnet two superconducting coils are mounted on top of each other and they produce a magnetic field similar to the way in which a Helmholtz coil would. The advantage to an open magnet 1204 is that it provides easy access to a subject 1210.

The magnet 1204 has a liquid helium cooled cryostat with superconducting coils. It is also possible to use permanent or resistive magnets. The use of different types of magnets is also possible for instance it is also possible to use both a split cylindrical magnet and a cylindrical magnet, although both are less convenient to use than an open magnet. A split cylindrical magnet is similar to a standard cylindrical magnet, except that the cryostat has been split into two sections to allow access to the iso-plane of the magnet. An open magnet has two magnet sections, one above the other with a space in-between that is large enough to receive a subject: as mentioned above the arrangement of the two sections is similar to that of a Helmholtz coil. Open magnets are popular, because the subject is less confined. Inside the cryostat of the cylindrical magnet there is a collection of superconducting coils. Within the magnet 1204 there is an imaging zone 1208 where the magnetic field is strong and uniform enough to perform magnetic resonance imaging.

On the inside of the magnet 1204 there are magnetic field gradient coils 1206 which are used for acquisition of magnetic resonance data to spatially encode magnetic spins within an imaging zone of the magnet. The magnetic field gradient coils 1206 are connected to a gradient coil power supply 1207. The magnetic field gradient coil is intended to be representative. Typically magnetic field gradient coils contain three separate sets of coils for spatially encoding in three orthogonal spatial directions. A magnetic field gradient power supply supplies current to the magnetic field gradient coils. The current supplied to the magnetic field coils is controlled as a function of time and may be ramped or pulsed. A subject 1210 is reposing on a subject support 1212 and is partially within the imaging zone 1208.

A surface coil 1214 can be seen as being on the surface of the subject 1210. The surface coil 1214 is a radio frequency antenna for manipulating the orientations of magnetic spins within the imaging zone and for receiving radio transmissions from spins also within the imaging zone. The surface coil 1214 is connected to a transceiver 1216. The radio frequency transceiver 1216 may be replaced by separate transmit and receive coils and a separate transmitter and receiver. It is understood that the radio frequency transceiver are simply representative. The surface coil is intended to represent a dedicated transmit antenna and a dedicated receive antenna. For instance, the magnetic resonance imaging system may also include a body coil for exciting magnetic spins. Likewise the transceiver may also represent a separate transmitter and receiver. The transceiver 1216 is a multiple channel transceiver it is connected to a magnetic resonance coil assembly 102 and the surface coil 1214. The magnetic resonance coil assembly 102 has been clamped around a fiducial marker 300. Other examples of magnetic resonance coil assemblies and fiducial markers may be used instead of those depicted. Additionally, more than one magnetic resonance coil assembly and fiducial marker may be used.

Within the subject 1210 there is a target zone 1218. A shaft or needle 1220 has been inserted into the subject 1210. The magnetic resonance fiducial marker 300 is on the shaft 1220. The magnetic resonance fiducial marker 300 is also connected to the transceiver 1216. The transceiver 1216 and the gradient coil power supply 1207 are connected to a hardware interface 1226 of a computer system 1224. The computer system further comprises a processor 1228. The processor 1228 uses the hardware interface 1226 to send and receive command signals to the magnetic resonance imaging system 1202. The processor 1228 is able to control the magnetic resonance imaging system 1202 via the hardware interface 1226.

The processor 1228 is further connected to a user interface 1230, computer storage 1232, and computer memory 1234. The computer storage 1232 is shown as containing magnetic resonance data 1240. The computer storage 1232 is further shown as containing a magnetic resonance image 1242 reconstructed from the magnetic resonance data 1240. The computer storage 1232 is further shown as containing a location 1244 of the target zone 1218. These are coordinates of the target zone 1218. The computer storage 1232 is further shown as containing magnetic resonance location data 1246. The computer storage 1232 is further shown as containing an image 1248 which has been rendered and shows the relationship of the shaft 1220 relative to the target zone 1218.

The computer memory 1234 is further shown as containing a control module 1250. The control module 1250 contains computer executable code for controlling the operation and function of the medical apparatus 1200. The computer memory 1234 is further shown as containing a location identification module 1252. The location identification module 1252 is able to determine the location of the magnetic resonance fiducial marker 300 using magnetic resonance location data 1246. The computer memory 1234 is further shown as containing an image segmentation module 1254. The image segmentation module 1254 is adapted for locating target zones, shaft entry points, and/or anatomical structures using the magnetic resonance image 1242. The computer memory 1234 is further shown as containing a rendering module 1256. The rendering module 1256 is used for generating the image 1248 using at a minimum the magnetic resonance location data 1246 and the location of the target zone 1244. The computer memory 1234 is further shown as containing an image reconstruction module 1258. The image reconstruction module 1258 contains computer executable code for reconstructing the magnetic resonance image 1242 from the magnetic resonance data 1240.

As part of the user interface 1230 a graphical user interface 1260 is displayed on a display device. Within the graphical user interface 1260 is an image 1262. This may be a magnetic resonance image or it may be an image which is generated. Within the image 1262 is shown the location of a subject 1264. Within the subject 1264 is a target zone 1268. There is a needle 1270 which is also shown with its position relative to the target zone 1268. The point marked 1272 is the shaft entry point 1272 of the shaft 1220 into the subject 1210, 1264.

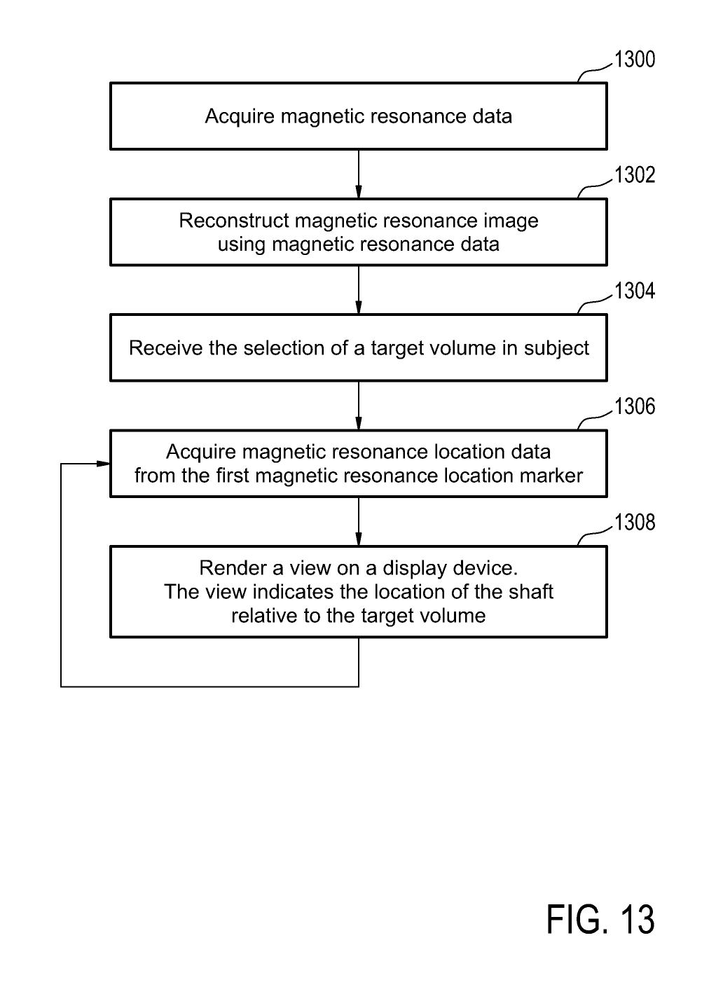

FIG. 13 shows a flow diagram which illustrates an alternative method of operating the medical apparatus shown in FIG. 12. In step 1300 magnetic resonance data is acquired. In step 1302 the magnetic resonance image is reconstructed using the magnetic resonance data. In step 1304 the selection of a target volume in the subject is received. This for instance may be performed manually and the selection may be received from a graphical user interface. In other embodiments the target volume is identified in the magnetic resonance image automatically using a segmentation module. Next in step 1306 magnetic resonance location data is acquired from the first magnetic resonance location marker. In step 1308 a view is rendered on a display device. The view indicates the location of the shaft relative to the target volume. In some embodiments the magnetic resonance image is also displayed on the view. Steps 1306 and 1308 are repeated during a procedure using an interventional device.

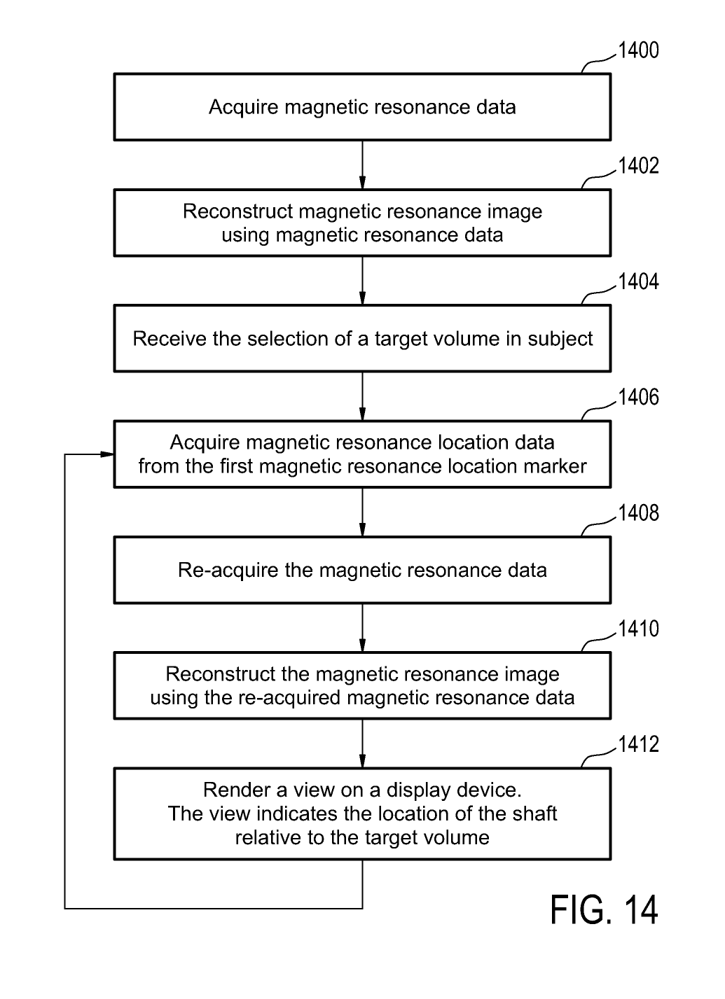

FIG. 14 shows a flow diagram which illustrates an alternative method of operating the medical apparatus shown in FIG. 12. In step 1400 magnetic resonance data is acquired. In step 1402 the magnetic resonance image is reconstructed using the magnetic resonance data. In step 1404 the selection of a target volume in the magnetic resonance image is received. In step 1406 magnetic resonance location data is acquired from the first magnetic resonance location marker. Next in step 1408 the magnetic resonance data is re-acquired. In step 1410 the magnetic resonance image is reconstructed using the re-acquired magnetic resonance data. In step 1412 a view is rendered on the display device. The view indicates the location of the shaft relative to the target volume and the magnetic resonance image is displayed as a part of the view. Steps 1406, 1408, 1410, and 1412 are repeated during a procedure using the interventional device comprising a shaft.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

### LIST OF REFERENCE NUMERALS

100 medical apparatus
102 magnetic resonance coil assembly
102′ magnetic resonance coil assembly
104 first clamping portion
104′ first clamping portion
106 second clamping portion
106′ second clamping portion
108 first antenna portion
108′ first antenna portion
110 second antenna portion
110′ second antenna portion
111 signal reception volume
112 antenna connection
114 elastic portion
116 pivot
117 handle
118 fiducial marker
200 perspective view
202 cross sectional view
204 MR signal emitting substance
206 encasing material
300 fiducial marker
302 perspective view
304 cross sectional view
306 hole
400 fiducial marker
402 perspective view
404 cross sectional view
406 removable plug
408 gap
500 fiducial marker
502 perspective view
504 cross sectional view
506 shaft
600 medical apparatus
602 clamp
604 first electrical contact
606 second electrical contact
700 medical apparatus
702 battery
704 transmitter
706 transmitter
800 medical apparatus
802 visual indicator
900 magnetic resonance antenna circuit
1000 medical apparatus
1002 perspective view
1004 hinge
1006 shaft
1008 alignment and fixation notch
1010 gap or opening
1100 top view
1200 medical apparatus
1202 magnetic resonance imaging system
1204 open magnet
1206 gradient coil
1207 gradient coil power supply
1208 imaging zone
1210 subject
1212 subject support
1214 surface coil
1216 transceiver
1218 target zone
1220 shaft
1224 computer system
1226 hardware interface
1228 processor
1230 user interface
1232 computer storage
1234 computer memory
1240 magnetic resonance data
1242 magnetic resonance image
1244 location of target zone
1246 magnetic resonance location data
1248 image
1250 control module
1252 location identification module
1254 image segmentation module
1256 rendering module
1258 image reconstruction module
1260 graphic user interface
1262 image
1264 subject
1268 target zone
1270 shaft
1272 shaft entry point

1. A medical apparatus comprising a magnetic resonance coil assembly, wherein the magnetic resonance coil assembly comprises:
a fiducial marker comprising a shaft of a medical device or is operable for receiving the shaft,
a magnetic resonance antenna comprising a first antenna portion and a second antenna portion for receiving magnetic resonance location data from the fiducial marker; and
a clamp, wherein the clamp comprises a first clamping portion and a second clamping portion, wherein the first clamping portion and the second clamping portion are operable for being moved between an open configuration and a closed configuration, wherein the first clamping portion comprises the first antenna portion, wherein the second clamping portion comprises the second antenna portion, wherein when in the closed configuration the first clamping portion and the second clamping portion securing the fiducial marker within a signal reception volume between the first antenna portion and the second antenna portion, wherein when in the open position the first clamping portion and the second clamping portion the fiducial marker is released into or out of the signal reception volume.

2. The medical apparatus of claim 1, wherein the magnetic resonance coil assembly further comprises a transmitter operable for receiving a magnetic resonance signal from the magnetic resonance antenna and transmitting it to a magnetic resonance imaging system.

3. The medical apparatus of claim 1, wherein the first antenna portion is a first saddle coil, and wherein the second antenna portion is a second saddle coil.

4. The medical apparatus of claim 1, wherein the first clamping portion comprises a first electrical contact connected to the first antenna portion, wherein the second clamping portion comprises a second electrical contact connected to the second antenna portion, wherein the clamp is operable for connecting the first electrical contact to the second electrical contact to form an electrical connection, wherein the first antenna portion and the second antenna portion are operable to form a single surface coil.

5. The medical apparatus of claim 1, wherein the magnetic resonance coil assembly further comprises a fiducial marker sensor system for sensing the fiducial marker.

6. The medical apparatus of claim 5, wherein the fiducial marker sensor system comprises any one of the following: a switch for sensing if the clamp is in the closed configuration, an impedance measurement system for measuring an impedance of the magnetic resonance antenna to determine if the fiducial marker is within the signal reception volume and/or determine a type of the fiducial marker, and combinations thereof.

7. The medical apparatus of claim 5, wherein the medical apparatus further comprises an indicator operable for displaying a signal if the fiducial marker sensor system senses the fiducial marker.

8-9. (canceled)

10. The medical apparatus of claim 1, wherein the clamp is operable for securing the shaft to the magnetic resonance coil assembly when in the closed configuration.

11. The medical apparatus of claim 1, wherein the fiducial marker comprises a hole for the shaft, wherein the fiducial marker is toroidal, and wherein the fiducial marker comprises a tube filled with a magnetic resonance detectable substance surrounding the shaft, wherein the tube has a gap, and wherein the shaft is operable for being removed at a right angle to the hole through the gap.

12. The medical apparatus of claim 1, wherein the fiducial marker comprises an adhesive for attaching to an object.

13. The medical apparatus of claim 1, wherein the medical apparatus further comprises:
a magnetic resonance imaging system for acquiring magnetic resonance data from a subject;
a medical device comprising a shaft, wherein the shaft is adapted for being inserted into the subject, wherein the fiducial marker is operable for being attached to the shaft;
a processor for controlling the medical apparatus;
a memory storing machine executable instructions for execution by the processor, wherein execution of the instructions cause the processor to acquire the magnetic resonance data, wherein execution of the instructions further cause the processor to reconstruct the magnetic resonance data into a magnetic resonance image, wherein execution of the instructions further cause the processor to receive the selection of a target volume within the magnetic resonance image, wherein execution of the instructions further cause the processor to repeatedly:
acquire the magnetic resonance location data from the magnetic resonance antenna wherein the magnetic resonance location data is descriptive of the location of the first magnetic resonance fiducial marker; and
render a view of the magnetic resonance data indicating the position of the shaft relative to the target zone on a display device, wherein the view is determined using at least the location data and the location of the target volume.
